(19) United States
(12) Patent Application Publication
(10) Pub. No.: US 2016/0175523 A1
(43) Pub. Date: Jun. 23, 2016

(54) AN INJECTION DEVICE FOR MEDICAL PURPOSES

(71) Applicant: Anders Blomberg, Kungsbacka (SE)

(72) Inventor: Anders Blomberg, Kungsbacka (SE)

(21) Appl. No.: 14/909,273

(22) PCT Filed: Sep. 11, 2014
(86) PCT No.: PCT/SE2014/000112

§ 371 Date: Feb. 1, 2016

(30) Foreign Application Priority Data
Sep. 17, 2013 (SE) 1300601-0

(51) Int. Cl. A61M 5/20; A61M 5/31; A61M 5/178

(52) U.S. Cl. CPC A61M 5/20; A61M 5/1782; A61M 5/31; A61M 2005/3114; A61M 2205/3584

(57) ABSTRACT

Injection device for medical use, adapted for injection by using a manual syringe. It can be inserted into a cavity in an inner tube (25), which is equipped with a restraint system for the syringe body (41), wherein the fixing and centering of the syringe at three levels are triggered by pressing down the syringe into the inner tube (25), through the depression releasing a spring-loaded surrounding intermediate tube (15) to protrude and enclose, fix and center the syringe (41). The spring force results from taking out the syringe when the enclosing middle tube (15) is pressed down against the spring force in an outer tube forming a housing (2) of the injection device, to a locked position. A displacement device (31) located in the inner tube (25) can displace the syringe body plunger portion (44) in two directions in a controlled sequence so that, during a loading step a selected amount of medication is automatically drawn into the syringe body (41) and after insertion in body tissue medicine in the selected amount is injected. The spring force resulting from the pressing down the protective middle tube (15) will, when taking out the manual syringe (40), then give power to the automatic protrusion of the centering and protective middle tube (15).

### TECHNICAL FIELD

For the treatment of certain diseases requires that the patient regularly, often daily, supplies medical materials by injection. One such disease, which affects a very large number of people, is diabetes, where it is important for the patients as far as possible to arrange for the delivery of insulin to the body on their own. However, in many cases the complications of this disease, including blindness constitute a complication. A large part of the insulin-treated diabetics also have other concurrent diseases that somehow impedes insulin injection, such as a lighter paralysis, rheumatism or parkinsonism. The fact that the diabetes rate also increases with age makes learning disabilities by charging and injection by means of a conventional syringe is a widespread problem. Now, insulin injection is usually performed with pre-filled insulin pens that are widely used in e.g. the scandinavian countries, an approach, however, that is more expensive than injection with vial and syringe. In some countries the cost element is important because the patient has to pay without other reimbursment. Often they commonly use two insulin pen insulin pens, one for slow-acting insulin and another pen with fast-acting insulin. Dosing occurs over the day with 1-5 injection sessions per day. A method for reducing the injection sessions is to use prefabricated mixtures with slow—respectively rapidacting insulin and injection times can be reduced to 1-2 times/day. To further increase the accuracy of the mixture, mixing can be done in a disposable syringe with a loading procedure that most perceive as complicated. Mixture of insulin is not possible in insulin pens except prefabricated mixtures in fixed proportions ex.: 50/50, 25/75 or 40/60. The diabetic who uses an entirely manual hypodermic syringe and is blind or disabled have considerable difficulties when using the prescribed amount of insulin and then performing insertion in the skin and injecting. It is previously known an aid in the form of an injection gun, which however only carry out the insertion. Some dispensing equipment are also available, but these are deemed by users as cumbersome to use. Insulin pens are definitely a great help but can not mix insulin. another patient group who requires the help of professionals with insulin injection, the multisick patient who, due to many physical and mental disabilities cannot make the injection itself.

Another group is injectionafraid patients, non-disabled people with diabetes as well as patients desiring close monitoring of its values, both given insulin injections and blood glucose measured values. Patients who wish to obtain statistical data in different form of their values and their injections performed, today has problems with the automatic loading of these variables.

### PRIOR TECHNICAL ART

It is through the Swedish Patent 8107458 to 5 (patent holder the same as applicant for this patent) known an injection device for disposable syringes. The older invention describes a motorized ejection of a protective and surrounding steel tube, syringe automatic locking and centering that now in this patent is done automatically by spring force created by removal of the disposable syringe. This patent describes a not previously known insulin recognition by coded vial holders. The object of the present invention is to make an injection device, wherein both loading of one or two types of insulin and insertion/injection is performed without the requirement of good vision or movement of the user. In addition insulin mixting is done in a simple and safe way. In addition to the electronics of the injection device makes it possible to maintain wireless communication between prescribers and users via bluetooth transfer. The advantage of this is after injection, also recorded laboratory values can wirelessly be reported to the healthcare provider. In addition, a prescribed dose can be changed wirelessly by the caregiver. This makes it possible to do without health care personnel at injections. The invention shall perform: a disposable syringe is depressed in the robot, whereafter a protective sheath metallic tube automatically raises and forms a protection around the disposable syringe. On the protective tube top an insulin vial is placed for loading of insulin to the disposable syringe to a pre-programmed amount. The vial is taken off and the robot is turned, pressed to the skin and is fired by pressing a trigger button. Subsequently the disposable syringe is taken out of the robot, which now waits for the next injection. The amount of insulin can be controlled wirelessly via bluetooth to the robot by remote prescription from doctors/nurses.

Said object is achieved by means of an injection device according to the present invention, whose characteristics are evident from the following claim #1.

### LIST OF FIGURES

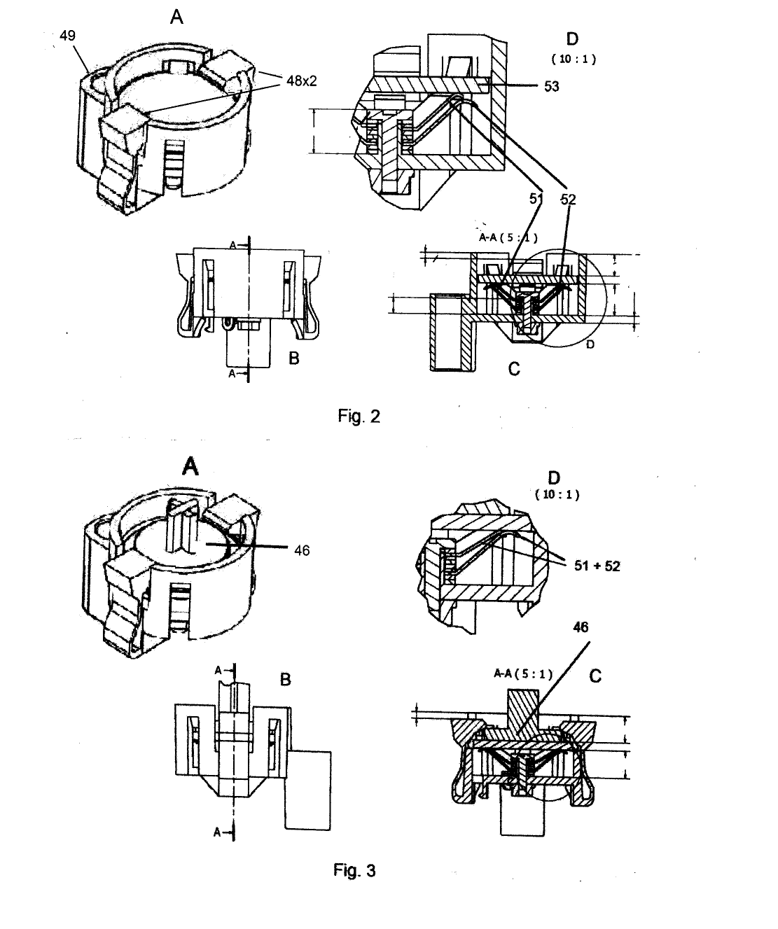

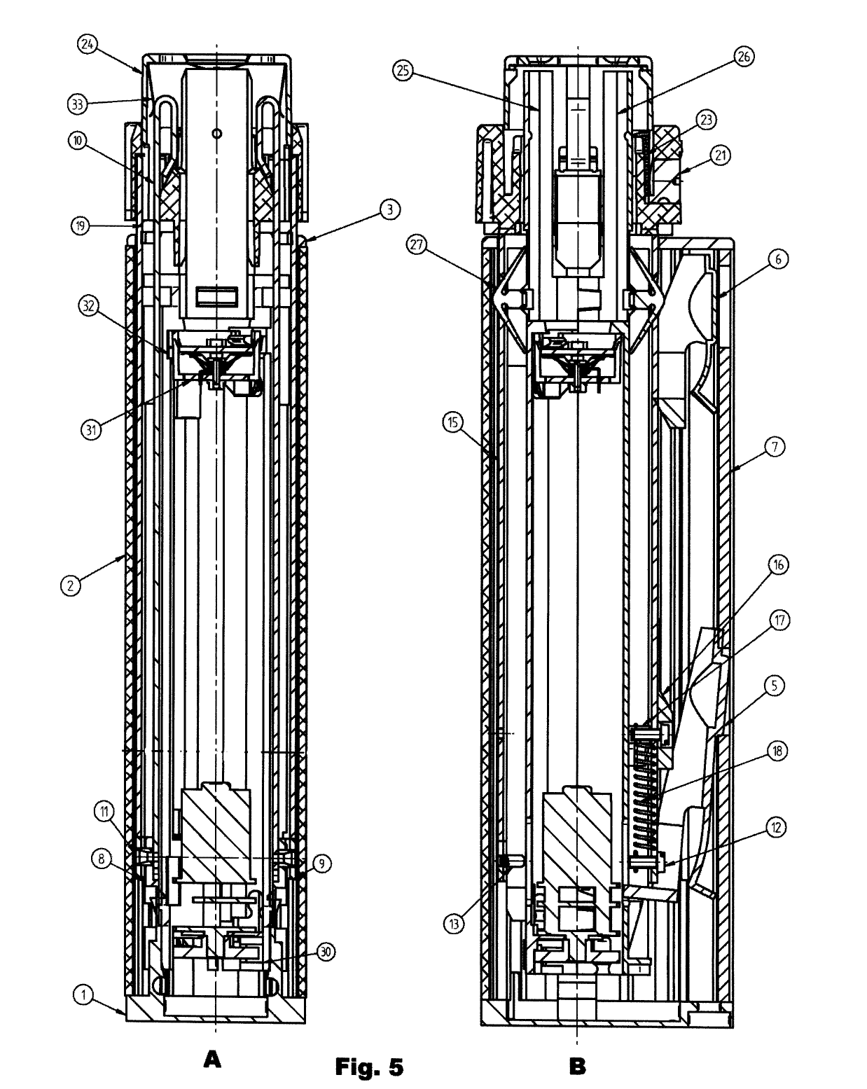

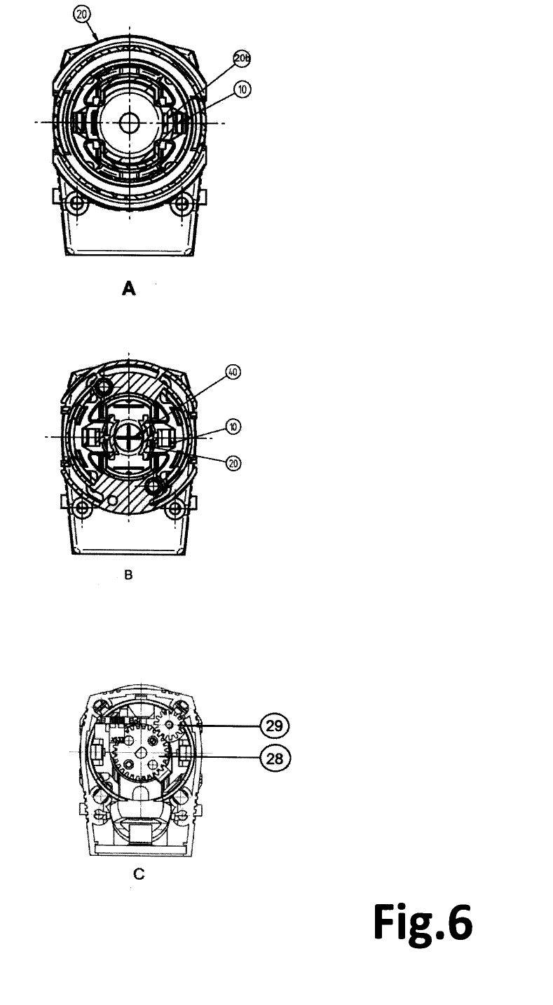

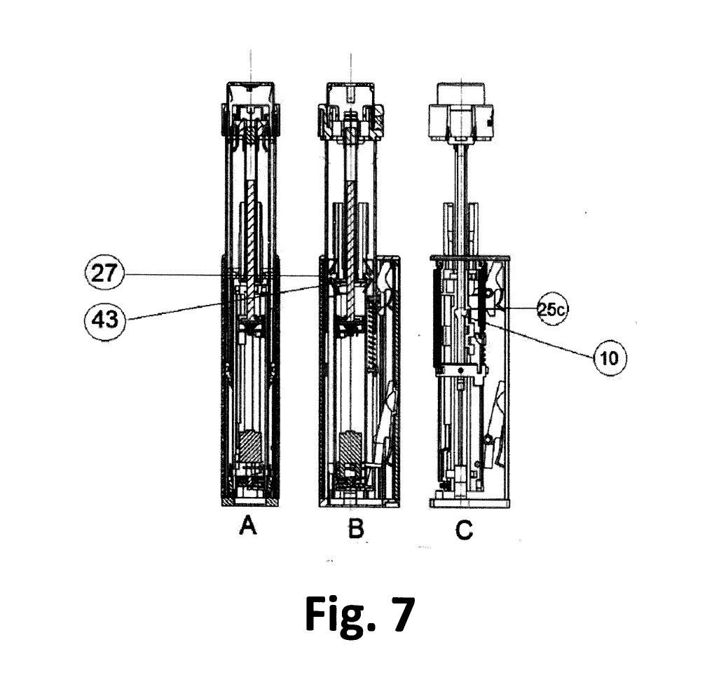

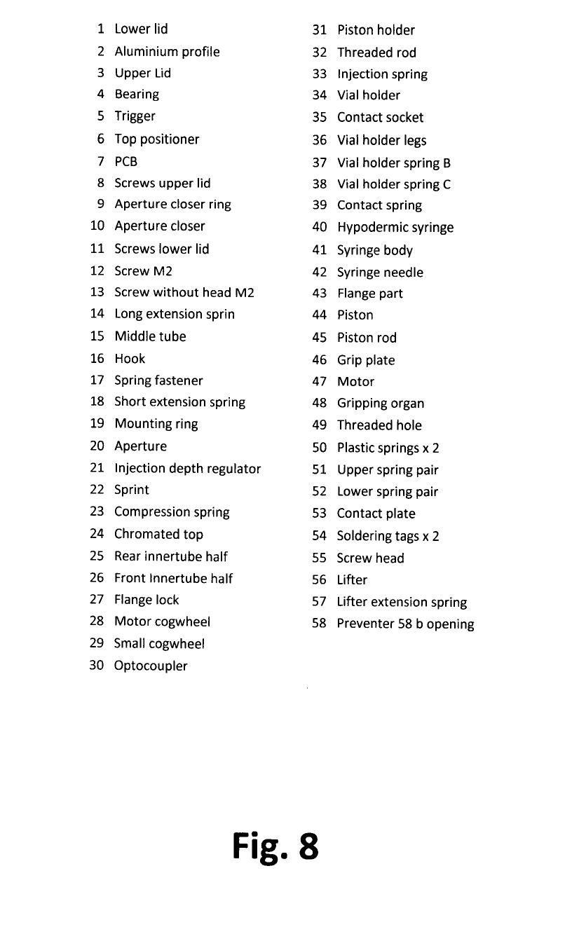

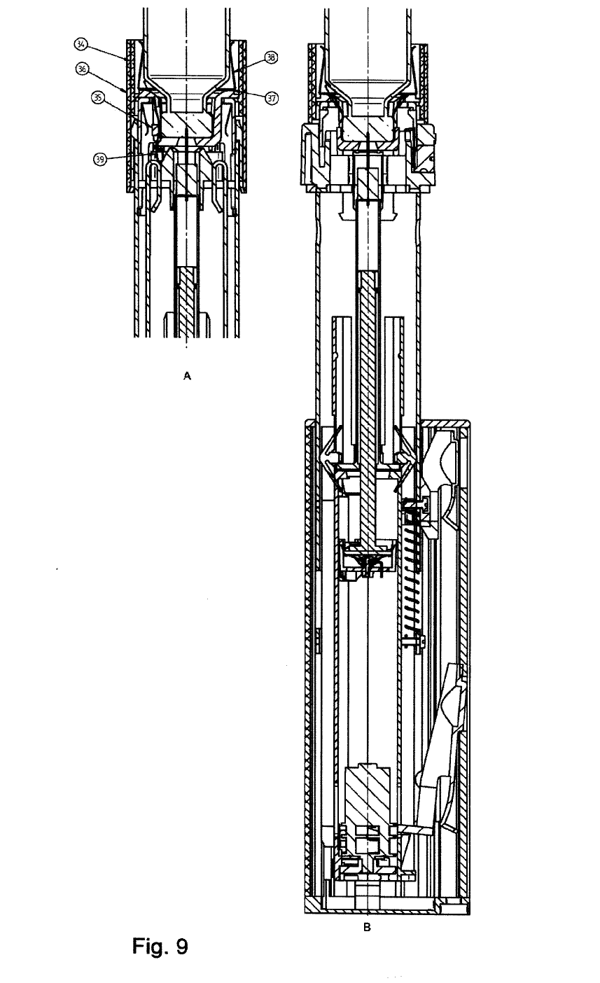

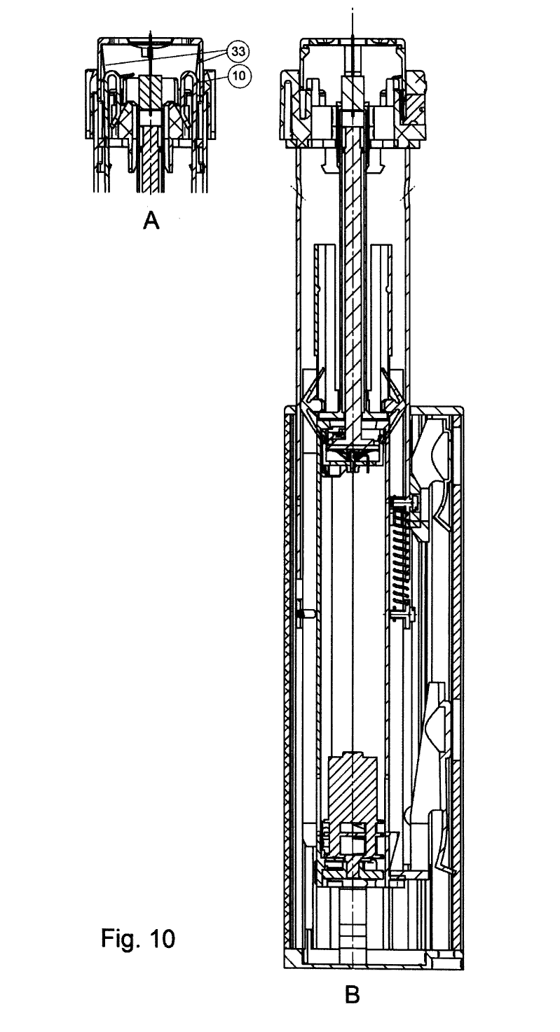

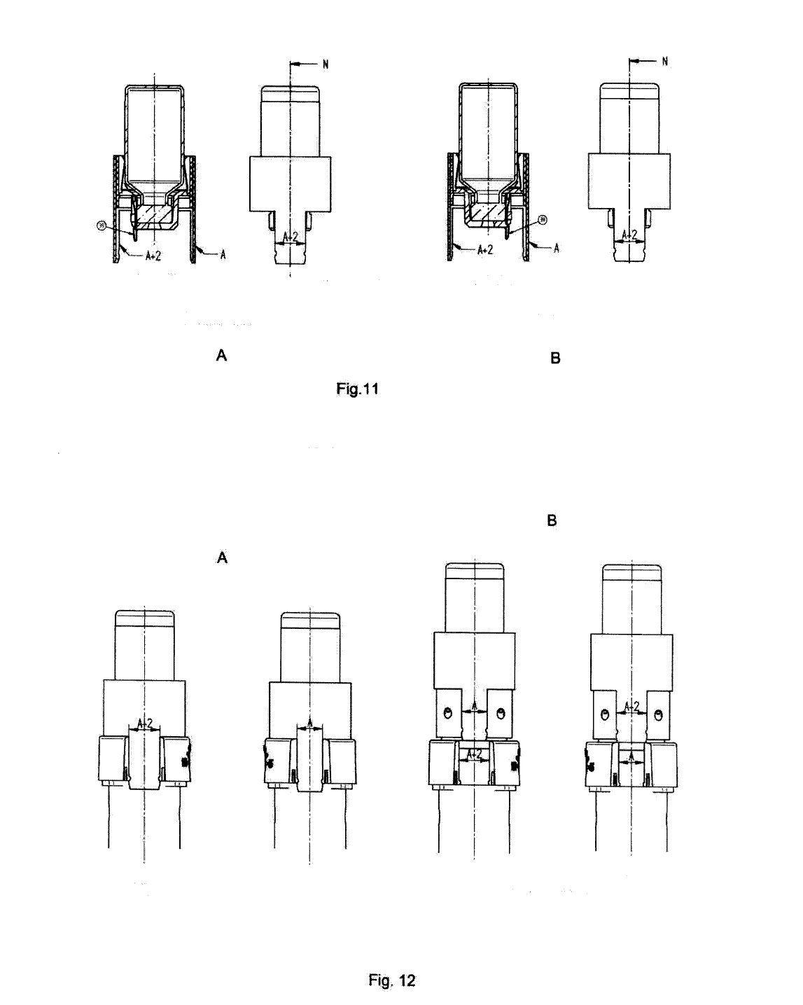

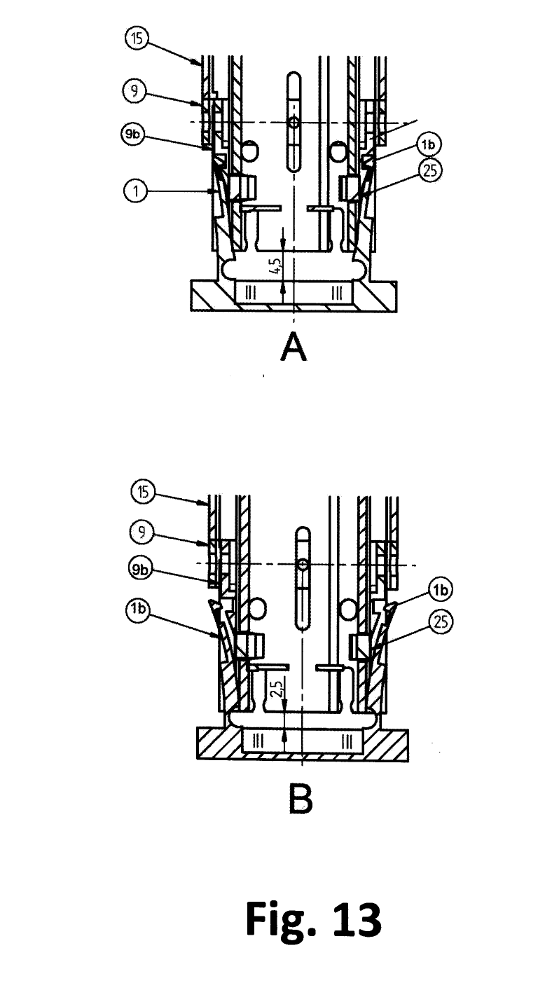

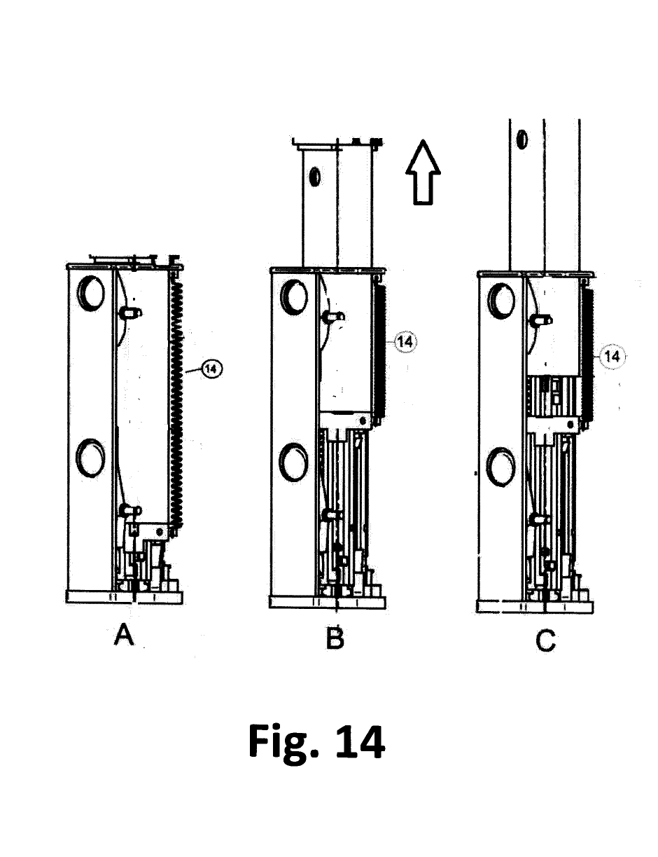

The invention will be further described with an example, with reference to the accompanying drawings in which position #1 (FIG. 5) shows the syringe in the sleep mode, pos. #2a (FIG. 7) in the extended position, pos. #2b (FIG. 9) with the loading of medicine and pos. #3 (FIG. 10), the syringe in the injection position with the injection of medical substances into a patient's body. FIG. 2-4 shows the piston holder attachment mechanism FIG. 6. a+b shows the centering mechanism of the syringe. FIG. 6 C shows the transmission mechanism. FIGS. 11 and 12 shows the coding scheme for the two types of insulin. FIG. 13 shows see attachment hooks. FIG. 14.15 o 16 shows how the spring system work automatically without engine power bringing the protective tube to the locked top position.

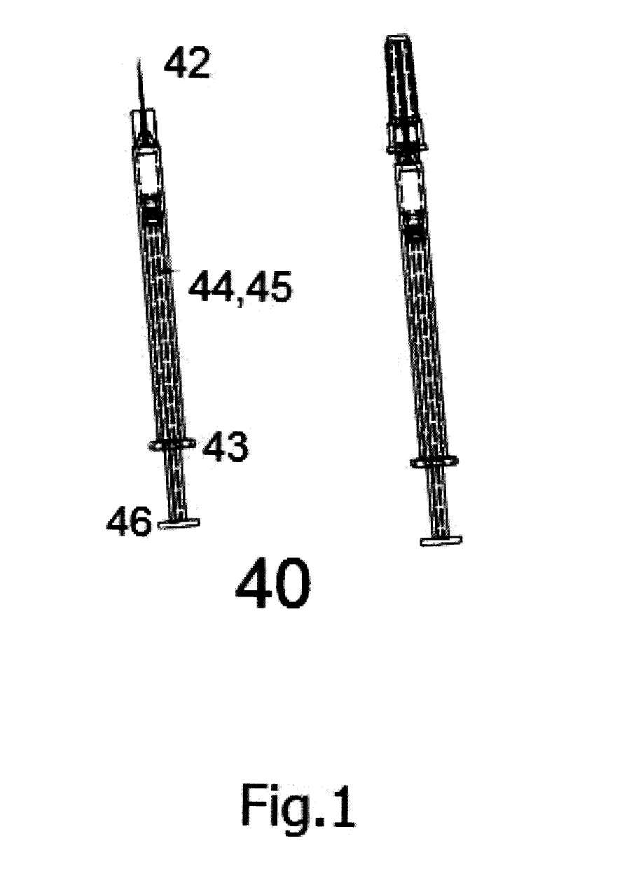

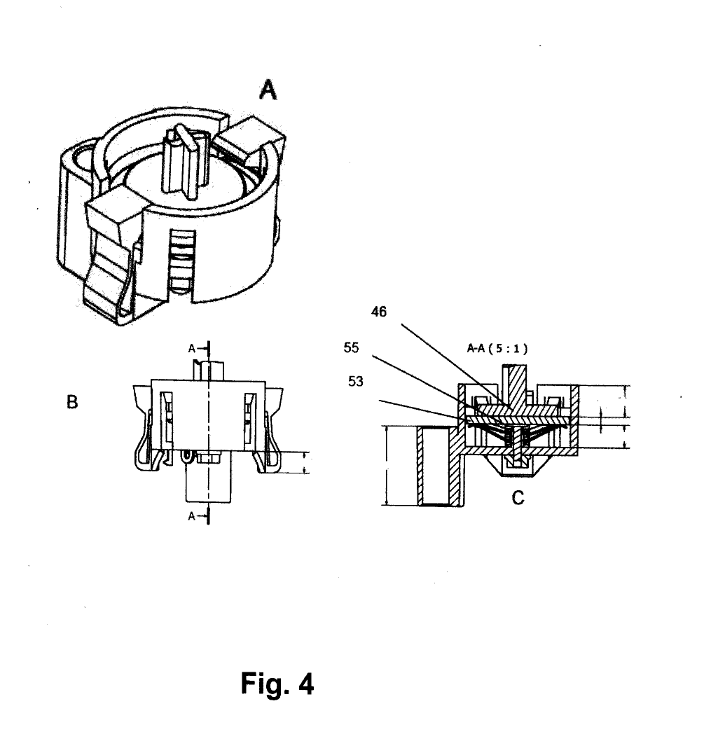

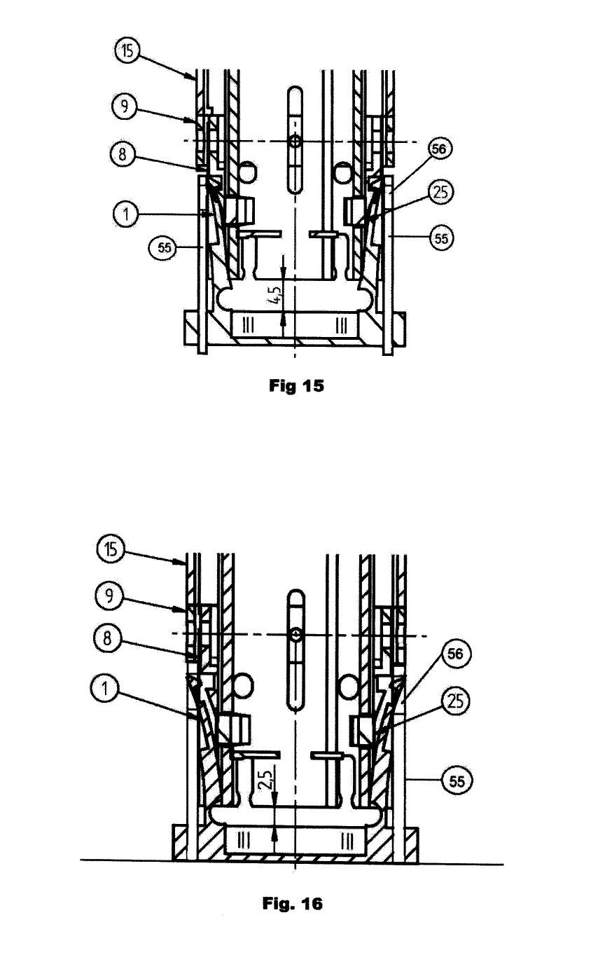

In the drawings, the injection device main characteristic is shown. This shows up at its upper end an opening through which a manual syringe type disposable syringe (FIG. 1) 40 can be inserted in a cavity in the injection device, The Syringe 40 is a standard product and substantially built up of a syringe body 41 that forms a cylinder and that at its upper end ha a needle tip 42 and at its lower end a flange portion 43 in the form of two side directed flanges. The disposable syringe 40 further has a piston part comprising a piston 44, piston rod 45 and one at the rods outer end a grip plate 46, by means of which the piston is movable within the syringe body to draw up end inject medical topics through the needle tip 42. The needle tip is covered by a needle guard mounted removable on the syringe body. The injection device (FIG. 5) is made up of three relatively each other displaceable main parts, namely an outer part 2, which forms a housing of the injection device, a relatively the house movable middle tube 15 and inside the middle tube a displaceable inner portion divided in two assembled halves 25 (a+b) together forming an inner tube in which the disposable syringe 40 is held in a manner to be described below. The housing 2 comprises a as a guide for the middle tube 15 a formed cylindrical bore, out of which the intermediate part can be pulled to an extended position pos. #2 (FIG. 9), which will be desribed later in the text. In the house is besides the middle tube 15 and the inner part 25 with associated interior also situated an electric drive motor and also a transmission mechanism FIG. 6. B. A larger cogwheel 28 drives a threaded rod 32 via a smaller gear 29 to provide the injection device/piston holder 31 to perform working movements. Further, the housing encloses an electronic control device, a circuit board PCB 7, adapted to control the drive motor certain pre-ordered movement. The PCB has five membrane switches, two of which one is intended for setting the fast—and one for slowacting insulin. Some patients take two different varieties of medication, can be mixed in the disposable syringe. Insulin amount of one kind is set thus by the key rapid and insulin amount for the other variety lente with the other key. The setting appears on the LCD screen. For simplicity, a case is presented where only one kind is set, which is the most common situation. The key that is designated regulates the desired quantity, entered with digital numbers on the display. The second variety can, for example, be set to a zero position. The electric motor is constituted in the example shown as a DC motor with a rotary output shaft which extends parallel to the middle tube 15 and the inner portion 25 ment movements. In he DC motor can be encapsulated an unshown gear mechanism to downshift the rotation. The transmission mechanism acc. FIG. 6C is constituted by a cogwheel 28 on the output shaft and a cogwheel 29 which interact with each other. The threaded rod 29 is rotatably mounted in the wall of the inner part 25. The inner part of both halves 25 (a+b) shows a displacement device 31 for the piston gripping plate. The displacing device, i.e. the piston holder 31 is displaceable in an inner portion 25 between an upper position, shown in pos. #1, and a lower position, shown in pos. #2. The piston holder 31 is formed with a substantially cylindrical body on which are attached two gripping means 48 adapted to firmly grip the grip plate 46 and move the piston together with the sliding movement of the piston holder. The piston holder 31 construction is best seen in FIG. 2 (Step 1) showing a broken partial view of the injection device in an enlarged scale in FIG. 5A and FIG. 2, piston holder 31 in FIG. 5A in its upper end position in which the grippers are situated in a releasing position, and in FIG. 3 in a downwardly displaced position. in which the grippers are in a gripping position to the grip plate 46, a position the gripping means (48×2) occupies during the piston holders movements except the position in the position shown in Fig. The piston holders 31 movements are produced by rotation of the threaded rod 29, which has attached a threaded hole 49, or more particularly from the cylindrical body 36 a projecting portion with a bore which is internally threaded. The grippers 48 are disposed in the cylindrical body and is pivotally mounted thereto by a respective plastic spring to the piston holder. By that the inner portion 25 in its wall has two oppositely located recesses intended to accommodate the grippers 48 of the piston holder 31 in the upper end position, make the grippers conversion to the release position by the action of plastic springs. Plastic springs tends to maintain the grippers in an expanded release position, thereby allowing the gripper plate 46 to be held in a free position. In the piston holder's interior part are two spring pairs 51 and 52 located, electrically isolated from each other. Above and resting on the highest spring pair is a metal contact plate 53 which are in electrical contact with the upper pair of springs. When gripping the syringe gripping plate the disc is pressed downward electrical contact is created between the gripper plate and the lower spring pair. This situation arises when the gripping plate 46 is in step 2 acc. to FIG. 3D and providing a contact between the two spring pairs. This contact function is relayed to two soldering tags 54 which are located just below respectively spring pairs 51 and 52. When there is contact between the spring pairs, it is also the contact between the two soldering tags. This contact function between the spring pairs gives a signal to the control circuitry on the circuit board that a disposable syringe is located in the device, which can be configured as a main switch for the injection device. According to step 2, FIG. 3, the contact between the spring pairs means that a syringe is located in the unit. When the syringe is removed and no contact exists between the spring pairs are the main switch off. In addition, there is a centrally located screw 55 FIG. 4C whose head is also a signal transducer. According to Step 3 FIG. 4 shows when the piston holder is moved upwards towards its highest position and the disposable syringe is locked, a top position is entered, the screw head is then an upper stop point for the piston holder's upward movement. Then the screw head 55 contacts the bottom of the contact plate. The screw head is connected to a third soldering tag. At the highest postion contact will occur between the screw head soldering tags and the other solder tags, a signal is conveyed to the PCB and it's electronic and processor. The processor receives this signal that the hypodermic syringe is in the most compressed position and all content in disposable syringe has been emptied. The injection device 1 of FIG. 5 further comprises an upper support device, called aperture, located in at the top of the the tube 15 just below the opening. The support means/aperture 20 is intended to form a support for the disposable syringe in its upper end and thus center it in the injection cavity and to off-hook automatically the needle guard, before the syringe loaded. The supporting device placement is shown in FIG. 5 and its construction is shown in FIG. 6 A. The supporting devise/aperture 20 is movable between a retracted position, shown in the pos. #1 FIGS. 6A and 6B, a forward support position, shown in the pos. #2, the retracted position is allowed disposable syringe to be inserted in place in injection device, while in the extended support mode the hypodermic syringe is centered and supported laterally. But the hypodermic syringe is not fixed, it is allowed to perform a displacement movement relative to the support device, which is performed during insertion/injection, as will be described below. The support device consists of two spring-loaded plastic wings 20b of the aperture, which are movable towards and away from each other on the inside of the middle tube 15. The two plastic wings are made of resilient plastic and are molded in one piece in the aperture 20. The spring action tends to maintain the supportive elements from one another in its retracted position. The supportive element includes two elongated tension parts=apertureclosers 10 in the form of electrical leading rails, which are electrically insulated from the middle tube 15 and also the other parts of the device. The two apertureclosers extends down to the lower end of the injection device in the FIG. 1 position and is firmly attached and connected with a aperturecloserring 9 which is slidable within the housing 14 relativevely both the housing and between the part 15 and the inner part 25. Aperturecloser ring is however coupled to the middle tube 15 via one or two tension springs 18 which are shown in FIG. 5 in the normal position and the tensioned position in FIGS. 7 and 9. This causes the aperturecloser ring 9 to accompany the inner tubes 15 outward movement during most of its travel distance, but is hooked by a hooking element 25 FIG. 7C caused by that the aperturecloser is enlarged to a cross and remains in that in FIG. 7 and FIG. 15 position, whereby both short-tension springs 18 in the middle tube 15 last motion will be tightened. As shown in FIG. 5, cut B-B with a partly broken portion the apertureclosers 10 are shown formed at the top as hook -shaped bulges. At bottom the aperture closers 10 are attached to the aperture tensioner ring 9. The aperture closer intermediate part has an enlargment, an abutment, which is hooked up in the inner tube 25. In this respect apertureclosers 10 stop its translational motion, whereby the middle part 15 and thus also the aperture 20 moves relative to the aperture closers 10. By that these (10) at the top are formed as respective obliqueformed guide surface, causes this a relative movement of the two plastic wings of aperture 20, to move against the action of its plastic springs toward the center, to the FIG. 7 B showed support position, thus producing disposable syringe 40 to be stabilized and centered. At the upper part of the inner part 25, two flangelocks 27 FIG. 5 B are provided which are holding device for the syringe body. The restraint formed by two spring-loaded wings and a central locking portion, forming a lock against which syringe 32 is tightened and it's two lateral flanges 41. In resting pos #1 the flange locks 27 FIG. 7b are non-latching. The top of the wings that form a V can come in a noncentered mode as the middle tube 15 has holes punched out where the top of the flanges locks V can be pushed through and then get in a non-latching position. When the middle part 15 is pushed upward the flange locks 27 are pressed inwards toward its spring action and form an interlocking edge against the syringe body flanges. When the middle tube at the end of the injection phases is pulled down and lowered the flange locks 27 will by its own resilient plastic wings be pressed out in the punched hole and allow that the syringe body flanges to become free.

The middle tube is pulled manually by the user after that a spring mechanism has been activated puling up the tube to a position just below the top position. The spring mechanism consists of two long extension springs 14, attached to the underside of top lid 3. The springs have their second hold in aperture tensioner outer ring. In resting position #1 the springs are maximally prolonged and the spring force is directed upward, tending to pull the aperture ring tensioner 9 upwards. This spring force is countered by two hooks 8, connected to the aperture tensioner ring FIG. 13 (S-S b). These hooks are latched in two upstanding hooks from the bottom cover 1. The hooks from the bottom lid fixes the hooks 8 and hold against the spring force from the long extension springs. Hooking mechanism is so designed that the bottom part hooks 1b has it spring force inwards and strive to lock the hooks 9b on both sides. Bottom part 1b hooks downward part is however conical with the largest opposing measurement at the top section. When the innertube 25 is pressed downwards FIG. 13 B in the conical boreof 1b that follow the inner tubes inner walls, then the hooks 1b from the bottompart are pressed out from the centre and the hooking mechanism releases. The spring force will then operate freely and the springs fastened in the aperture tension ring will move upwards as well as the middle part/steel tube 15. The tube 15 is now almost in the top position FIG. 15. The long extension springs which are secured in the aperture tensioner ring 9 can not bring the tube 15 further up. To get the tube 15 to come to it's top position named pos #2 the tube 15 must manually be pulled up further some centimeter. Pulling up the middle part/tube FIG. 7 ends with that the top positioned 6 engage a projection 16 fixed on the tube 15 and fixes the tube in the top position. When the whole injection is finished, the hypodermic syringe is taken out by pushing down the middle tube to it's bottom position so that the hooks of the lower lid again locks the hooks in the aperture tension ring. The middle tube is thus pressed down against the force of the long extension strings. This force can then be used when pulling out the middle tube at start of the device. It is a semi-automatic course of events by finally manually pulling out the tube 15, but first by spring force the middle tube 15 and aperture tension ring 9 jointly is pulled up by the extension springs 14 secured in the aperture tension ring 9 up to a position where the last short pulling out is made manually by pulling the middle tube upwards by gripping the aperture. During this last pullout the brief tension spring 18. is set under tension. The short extension spring 18 then acts as a force giver to the hypodermic syringe's insertion in the skin, the whole inner tube 25 with fixed disposable syringe jumps by spring 18 force so that the insertion occurs.

The now described movements describe the middle tube's automatic expulsion, the inner tube has not deviated from its position in pos #1 more than the small depression at some mm, affecting the bottom's hooks to release the aperture tension rings hooks so that the tension ring freely slide up. Now we shall describe how the inner tube with fixed hypodermic syringe performs the insertion, ie. inserts and move relative the intermediate tube 15 and the aluminium house 2

To avoid that the latching mechanism releases the ejection of the middle tube in an unplanned manner without insertion of a disposable syringe, a security system can be put in place to hold the holding hooks, by e.g sharp shakings. Two longitudinal vertical barrings 58 FIGS. 17 and 18 are located on the outside of the locking hooks on the bottom lid. They are resilient so that the latch is pulled downwards. In the top of the latches is an opening 58b located. When the injection device is set on a plane, the latch moves upwards because of the latches lower parts parts are located beyond the base in free mode FIG. 17. When the injection device is freely located the latch prevents the hooks to extend FIG. 17. When the latch is placed on a plan FIG. 18, the latch is pushed up and a cavity provides space for the hooks to expand when a syringe and the internal parts are pressed down.

Referring again to FIG. 5 B shows that the inner part is held in its FIG. 1 position by a trigger 5 which is pivotally mounted on the housing and can be activated by the pressure of the finger with rebounding force of its own plastic spring. The trigger 5 interacts with a protrusion at the bottom of the inner part 25 as long as the trigger 5 is not depressed. The upper top positioner 6 shows a corresponding mechanism, which also is pressed in against the spring action of the plastic, which occurs when the middle part 15 is to pushed down to pos. #1 This is done by hand after injection device has completed it's working operations. Further, the injection device on top of the aperturte 20:01 a button, the depth setting button 21 which has a concentric pivot regulating different possible depth of insertion by rotation. Within the aperture 20 is located a chromated top cover 24, which is the opening/entrance for the syringe to the device. It is under spring tension such that it strives to assume a position in #1 showed elevated position, but can be depressed as long as the syringe is in its inner position. Upon depression of the chromate top cover 24 an electrical circuit is closed that will activare the motor for injection, which is accomplished by means of contact plates, called the injection spring. The injection spring provides electrical contact between the two aperture tensioners and electrical ground, effected by that the tube always provides electrical earth as well with electrically connected metallized top cover.

The circuit board PCB 7 is not shown but has multiple options buttons as: starter/reset button and memory keys. Most important are the setting buttons Rapid and Lente, which are electric push buttons connected through the controller to an LCD screen. After setting is done with buttons/LCD, insulin vial with holder are attached to the top and the required amount of insulin is withdrawn from the vial. The drive motor is fed back by an optical encoder 30, which returns the information to the microprocessor about the number of turns of the motor and hence the work done. The optical encoder is constructed with a frequence-sensor that a feedback signal communicates a frequency corresponding to the number of pulses for each unit of insulin, for example 10 pulses per unit. Insulin doses are are commonly prescribed in units, 10 units equivalent to a tenth of a milliliter, commonly referred to as a line, as disposable syringes are graduated in such marking. To the microprocessor and the feedback circuit is also connected a buzzer that marks the number of units drawn aspirated during the loading operation, for example, every unit and higher pitched signal at every 5th unit. On the input side to the microprocessor two switches are attached via time delay and adaptation circuits, connected in the injection device so that certain operations start by electrical activation. A switch has been previously mentioned is situated in the piston holder 31 and is closed by insertion of the syringe device, more particularly by depression of the grip plate to press down the contact disk and the two described spring pairs. The second switch has also been mentioned above, and signals the when the disposable syringe is in place and withhold by the piston holder. The switches are then responsible for further report to the microprocessor that a syringe is in place, also tell when the syringe is compressed and in reverse mode, further on that the insulin vials are put on for loading and finally that the syringe-needle is inserted and can start the injection mode. All of this is found in the processor circuit records and reported to the data server periodically through radio (Bluetooth/wife) transfer to PC or mobile phone, which acts as a relay station between the injection device and the server computer. The mobile phone can also describe how the injection process has gone and can decipher the signals of mishandling located in the registry. In addition, report amount of residual insulin in the vials and the charge state of the rechargeable battery. Being able to have a record of residual insulin in the vial is made by that each new vial insertion is marked by pressing a special key combination. Such a register is desirable because visually impaired people can not notice the amount of residual insulin. A third switch attached to to the aperturetensioners 10 which each is electrically connected separately to the circuit board. Aperturetensioners 10, thus, besides the mechanical function as centring the syringe, has an electrically scanning function, contact with the electrically grounded between middle tube can in various ways provide signals to the electronics. The signals are activated when an insulin vial is inserted into the top of the middle tube or when the top cover is depressed and the trigger is pressed simultaneously, wherein the electric motor is activated for injection.

The injection device can distinguish between types of insulin. In a vial holder for rapid-acting insulin is a contact spring installed in the contact socket FIG. 11 B (#2 insulin vial holder) to the right and gives contact with the right aperture tensioner, thus reporting that insulin vial of rapidacting insulin is on. In the same way 11 A way the vial holderr for slow-acting insulin is encoded by the contact spring located on the left side and thus makes contact with the left aperture tensioner. It is impossible to turn the bottle holder 180 degrees, thus allowing contact with the wrong aperture tensioners. It is impossible because the bottle holder has two protruding bones with different widths. These legs fit into recesses on the outside of the aperture. If you try to put the vial holder on in wrong position see FIG. 12, B, it does not allow the different widths of vial holder legs to fit to the aperture. A 180 degree mishandling makes that insulin loading can not be implemented. Thus, the robot can distinguish between different types of insulin, provided that the vial at the first time is put into the correct vial holder. Distinguishing types of insulin is important especially when mixing two insulins, a process difficult for non-medically trained. These coded insulin vial holder makes insulin mixing in the same syringe easy and perform without mistakes. Insulin aspiration from one vial is simple, only one insulin bottle vial on and the preset amount is withdrawn from the vial. However, this requires further explanation. First action is that the hypodermic syringe after insertion into the injectiondevice aspirates the same amount of air into the syringe as the set quantity. This must so be be due to that insulin not at all loading times can be pulled out from the vial, then a vacuum is created. First, you must inject air corresponding to the desired amount of insulin injected into the vial, then the amount of insulin is asåirated and later to be injected. When two types of insulin are prescribed, insulin mixture in the syringe procedure becomes more complicated. The two insulins are set via push buttons and LCD. After disposable syringe have been inserted, is it loaded with air equal to the sum of the two insulin doses set. Then the rapid insulin vialholder is attached. Air is injected in rapid insulin (#1). Then slowacting insulin (lente) vial holder (#2) is attachedand air is injected into slow insulin, thereafter aspiration of lente insulin. Then rapid insulin is loaded again (#1) and insulin is aspirated from the already air-injected vial. This behavior thus implies 3 vial holder attachments although only two insulins are set. It's venting problem that makes 3 attachments necessary. The insertion/injection is then made as in the same way as with single insulin injection. This insulin mixing process of two simultaneously set insulins are often difficult to teach and difficult to perform, but this robot performs the process easier by the robot recognizes insulin type and gives an error signal when not doing correctly.

Referring to FIG. 5, pos. #1 to FIG. 9 pos #3 is now described the various stages by the use of the injection device. The injector start position, resting mode is shown in FIG. 5, pos#1, before any disposable syringe has been inserted in the device. Hereupon, the middle tube 15 is situated in it's fully inserted/downward position. The piston holder 31 is in it's most upper position, thus the grippers are in the releasing position. Since the tube 15 is in its inner/down position the flange locks 27 are in the released position as well as the support device/aperture 20 which is in its retracted position. All three switches are hereby open. When a disposable syringe 4 is inserted into the injection device through the opening until its flange portion 43 passes the flange locks 27, the contact plate is depressed against the upper spring pair of displacer/piston holder 31, wherein the electrical switch is closed, the drive motor is started in such a direction. that the displacer/piston holder moves inwardly ie downwards. Simultaneously the gripping hooks 48 move immediately to the retaining position by the pistonholder/displacers movement downwards, whereby the gripping disc of the hypordermic syringe is made to move in a downward direction and so also the plunger is displaced downwardly. Pos #2 shows this mode, when the middle tube 15 is lifted. That the piston is entrained and the displacer/piston holder 31 is moved downward in this first movement, however, has only the function to aspirate air into the syringe, since loading of insulin is not yet started. This first movement is only intended to eject the middle tube 15 and bring with the elongated the aperture closers 10 together with aperture closer ring 9, attached to the middle part by the tension spring 18. A short position before the upper end position of the middle tube 15 the aperture closers 10 are hooked up and thus also the aperture closure ring 9 by stopping edges in the inner part 25, wherby the aperture closer ring and aperture closer stop their upward movement, while the middle tube 15 continues it's last movement to the top position by manual pull-up In this movement the aperture 20 with it's both plastic wings move from its retracted position to its extended/centered position for centering of the hypodermic syringe through interaction with the two aperture closers 10 oblique edges. During the movement of the middle tube 15 movement which is done automatically by force from the long tension springs, even the restraint system of the flange locks 27 move from their released positions to their hooking position. In its outer/upper position top postion the middle tube is held by the top postioner 6 and remain thus in this mode. Herein moves the piston holder/31 again to its in FIG. 7 showed lower position, which is the starting position for moment of loading This position corresponds to the push buttons by Rapid/Lente insulin preset volume. A mainly circular detachable vial holder is attacheed outside the top cover 24, having a circular opening through which an inverted insulin bottle is inserted so that the syringe tip may penetrate through the rubber stopper of the vials. At this moment the contact spring 39 get's in electrical contact with one of the aperture closers corresponding to the desired insulin injection. The contact spring 39 FIG. 11 is arranged so that insulin #1 has contact spring 39 disposed to the right and insulin #2 is located to the left. The circular vial holder has two legs with different width, A, and (A+2) suitable to be applied to aperture only one way. The wider leg (A+2) can not be applied to the robot more than one way, the more narrow track on the robot aperture does not accept the wider leg (A+2) FIG. 12. This makes the contact spring's 39 location to the left or to the right right tells the robot what type of insulin that is inserted at the top. When the electrical contact between the contact spring and aperture tensioner 10 is created the robot knows what kind of insulin that is attached. In this way the microprocessor is activated to start the drive motor so that the displacer/piston holder 31 is moved upwardly to its upper end position, thereby the vial will be filled with the amount of air equal to the amount of insulin to be injected. When the piston holder 31 has reached its most upward position the the piston has reached its upper position, the drive motor reverses direction, then the displacer 31 goes to the programmed/set value shown in FIG. 11 and the disposable syringe 4 is filled full with air. Insulin vial is attached, needle penetrates the vial's rubber membrane and the piston is pressed upwards in the syringe towards the bottle for injecting air into the vial, whereafter the syringe goes towards the upper end position meaning compressed syringe, then activating contact for compressed syringe and the control circuit reverses the motor direction and moves the piston with the piston holder 31 to a position corresponding to the set amount of insulin, the amount which therefore now is in the disposable syringe 40. Loading has now been completed, so the insulin vial attached vial holder is lifted off.

Appropriate insertion depth is set by the button for insertion depth. Hereafter the injection device with the top cover 24 is pressed toward the body tissue into which the insulin to be injected. Top cover 24 is pushed against the action of its spring. The device is now ready for insertion/injection FIG. 9, which is done by pressing the trigger button 5, wherein the action of the tension springs 18 move the inner part 25 together with the syringe is inserted in the body, until the two legs of the injection spring 33 comes in contact with both the aperture tensioners 10. The syringe unit with its spray tip thus penetrates to the required depth by closing the circuit between the two aperture tensioners to earth through injectionspring's legs 33, injection occurs. ie that the drive motor moves the displacer/piston holder 31 and thus the hypodermic syringe 4 piston 10 forward so that the contained amount of insulin can be injected into the body tissue. At the same time, preferably a buzzer sound during the entire injection torque. When the piston holder has reached its end position and the entire amount of insulin has been injected, the injection device is placed on a surface, and the syringe is taken away. The piston holder 31 is then in its upper released position. The flange locks 27 are also in their release position.

The invention is not limited to the above described and in the drawings shown embodiments, but can be varied within the scope of the following claims. Values from a blood glucose meter can be transferred via Bluetooth to the injection device to forwarding to the database. The injection device can also be used as electronic syringe pen, i.e. that repeated inserts/injections can be done without repeated loading from insulin vials. Through a set combination of two buttons on the push button panel the entire syringe is fully loaded, to it's maximal volume, in the present model with 100 units of insulin. The same loading can also be done at the request of a program performed on a bluetooth connected computer or cell phone, which then transmits the command to the injection device which after maximum filling is fully loaded with insulin. Next step is to pull the trigger on the device now standing in pos #3 prepared for injection but without depressed top cover.

Injection doesn't occur immediately but at every supposed injection/occasion an alarm buzzer sounds that an injection should be given and the patient takes the injection device to the skin without pressing the trigger. The injection device/top cover is pressed against the skin and through the activation/grounding of the injection spring against the two aperture closers 10, the injection occurs automatically and can be repeated on requested times until the withdrawn amount of insulin has ended in the the syringe and reloading must occur. This procedure is suitable in home care for elderly insulin dependent patients. Home healthcare personal can then load into the syringe full and programming time/amount of insulin via the internet through Bluetooth/data server. Only action the patient needs to do is to bring the injection device to the skin. Receipt of completed injection is sent via bluetooth connection. If the injection is not made at the requested/programmed time, a message will be sent to the caregiver that the expected injection has not occured. Similarly, the injection device will alarm if the remaining amount of insulin is not sufficient for the next subsequent injection. To avoid this problem to occur, two injection devices can be fully loaded with insulin at the same time and when one injection device is empty of insulin, the other device is activated and pursue the coming injections. This control over the remaining insulin is controlled by software in the injection device with Bluetooth transfer or mainly by programs in cell phone with the same type of transmission.

1. An Injection device for medical use, adapted for injection by using a manual hypodermic syringe, which comprises partly a syringe body (41), one end of which is located a needle tip (42) and whose other end has a flange portion (43), and and a piston part within the syringe body as a slidable piston (44), at whose movements medicine may be sucked in and pressed out from the syringe body, and with the piston rod with the piston connected grip disk (46), the device is constructed of three relative to each other movable head parts, namely an outer part (2) forming a housing, a relatively the house displaceable intermediate part in the form of an intermediate tube (15) and in the inside of the tube a displaceable inner part in the form of an inner tube (25), which has an opening through which the syringe can be at least partially inserted into a cavity in the inner tube (25), including a syringe body restraint with flange locks (27) shiftable between a holding position and a released position for the flange portion mechanically performed by the intermediate tube by pressing the flange locks inward to the restraint position for flange portion to be restraint and compression of the flange locks by spring action outwards to release the flange portion, when the middle tube's inwards pressing influence ceases, one in the middle tube situated upper support and centering device (20) for supporting and centering in the upper end of the syringe into the injection device and movable between a retracted position and an inwards support position for the syringe, a displacement device (31) adapted for displacement of the grip plate (46) and thus the pistonpart between an upper and a lower displacement position and adapted by gripping means in the top position being in a piston grip disk (46) release position and that in the lower displacement mode to be in a position for gripping the grip plate and following it in the displacer displacement movement, whereby the middle tube's (15) upper end is adapted to be attached to a removable vial holder (34) for a vial with the medicine to be injected during a loading moment and medicine can be drawn into the syringe body and the inner tube is movable in the middle tube to cause insertion of the needle tip in a body tissue, wherein the housing is located an electric drive motor and a transmission mechanism controlled by an electronic control device for controlling the motor which, via the transmission mechanism is coupled to the displacement mechanism to achieve it's displacement movements by the control device controlled sequence so that, during the loading sequence a chosen amount of medication is automatically drawn into the syringe body and after insertion into the body tissue medicine the chosen amount is injected, the insertion motion is generated by a first spring mechanism in the form of a tension spring (18) coupled between the middle tube and in an outer tube movable, following most of the movements of the middle tube, in in one position hooked tension ring (9) releasable from hooking with the housing attached manually operable trigger (5), said elongated tension organs (10) is fastened in the clamping ring and arranged to accompany their movement in order to change the support device (20) from releasing to supporting, characterized by that a second spring mechanism in the form of tension springs (14) are fastened with one end at the top of the housing and with a second end of the tension ring (9) and adapted to be in tension state by pressing down of the middle tube and thus also the inner tube to be latched by latching hooks arranged so that further pushing down of the tube, in a later sequence when depressed by the syringe, is converted to a position releasing it to allow the outpulled tension springs with its spring force to lift the pipe through the tension ring.

2. An injection device according to claim 1, characterized by that the second spring mechanism (57) is arranged to bring up the tube to a position where the first spring mechanism (18) is adapted to be tensioned by manually pulling out, thereby producing a spring force on the inner tube (25) which provides for the movement, making the insertion movement of the needle.

3. An injection device according to claim 1, characterized by the release of the bottom lid's hooks (1 b) and thus the upward movement of the tube (15) is prevented by a position sensitive mechanical hinder (58), so that only a vertical position of the injection device standing on a horizontal allows the release of the bottom lid's hooks.

4. An injection device according to claim 1, characterized by that the elongated tension organs (10) are electrically conductive but electrically insulated from each other and each individually electrically connected to the control device, for each of a user chosen medicine vial, the vial holder is specially encoded for one medicine kind by unique positions of electrical contact means (39) in the vial holder and the vial holder and the upper end of the middle tube exhibits mechanical code organs, arranged to allow only one way of applying the vial holder to the middle tube, whereby the vial holder's contact organ (39) can come in contact with the chosen tension organs (33) in the middle tube's upper end, and thus through the one or the other tension organ give a signal for the steering control to recognize the vial holder and thus type of medicine.

5. An injection device according to claim 4, characterized by that the mechanical code organs (39) are designed as projections from the vial holder and fitting in a determined position/recess in the middle tube's upper end or that the recesses are in the vial holder and the projections protrudes from the tube.

6. An injection device according to claim 4, characterized by that contact organs (33) attached to the upper end of the middle tube are arranged to also recognize when the syringe reaches the set penetration depth to complete the injection.

7. An injection device according to claim 1, characterized by an electronic connection via wireless communication such as Bluetooth connectivity can report the type and amount of drug and the time of injection to and from the injection device via the Internet to a communication center that is monitored by the prescribing medical staff.

8. An injection device according to claims 4 and 7, characterized by that after a full filling up of the syringe to its maximum volume with a type of insulin, injection device can at many occasion inject from the same filling and be used as a pre-filled electronic insulin pen that automatically without manual button activation injects the via Internet prescribed dose by the help of the above-mentioned contact means (33) in the middle tube (15) and is activated only by the pressure of the injection device to a patient's skin.
